(19) United States
(12) Patent Application Publication
(10) Pub. No.: US 2025/0186255 A1
(43) Pub. Date: Jun. 12, 2025

(54) Image Processing Based Vacuum Surge Detection

(71) Applicant: JOHNSON & JOHNSON SURGICAL VISION, INC., Irvine, CA (US)

(72) Inventors: Assaf Govari, Haifa (IL); Andres Claudio Altmann, Haifa (IL)

(21) Appl. No.: 18/533,473

(22) Filed: Dec. 8, 2023

(51) Int. Cl. A61F 9/007; A61B 3/14; G06T 7/00

(52) U.S. Cl. CPC A61F 9/00736; A61B 3/14; G06T 7/0016

(57) ABSTRACT

A phacoemulsification system and method, the system having a probe with a needle at its distal end, the needle configured to be inserted into a patient's eye, the probe having an ultrasonic transducer; an aspiration line fluidly coupled with the needle; an AVS system configured to control fluid flow in the aspiration line; and a processor, configured to: obtain a first set of images having a first image depicting a part of the needle and a particle of a lens of the eye occluding an aspiration line of the needle; obtain a second image depicting the part of the needle, the second image captured later than the first image; determine based on the first image and the second image whether an aspiration-stopping criteria is met; and subject to the aspiration-stopping criteria being met, control the AVS system to restrict fluid flow along the aspiration line.

### FIELD OF THE DISCLOSURE

This disclosure relates to detecting sharp changes in the vacuum generated in an aspiration line during a phacoemulsification operation, and avoiding resulting damage to the eye.

### BACKGROUND OF THE DISCLOSURE

A cataract is a clouding and hardening of the eye's natural lens, which often happens when people get older. A common treatment of a cataract is phacoemulsification cataract surgery. In the procedure, a clear cornea or scleral incision is made in the eye to access the internal structures and then a portion of the anterior surface of the lens capsule is removed to gain access to the cataract. The surgeon then uses a phacoemulsification probe, which is an ultrasonic handpiece with a needle and a coaxial sleeve at least partially surround the needle. The tip of the needle vibrates at ultrasonic frequency, which emulsifies the cataract lens. At a same time, a pump aspirates particles and fluid from the eye through the tip, wherein the aspirated fluids are replaced with irrigation of a balanced salt solution via the sleeve to maintain the intraocular pressure in the anterior chamber of the eye. After removing the cataract with phacoemulsification, the softer outer lens cortex is removed with suction. An intraocular lens (IOL) is then introduced into the empty lens capsule restoring the patient's vision.

### BRIEF DESCRIPTION OF THE DRAWINGS

The present disclosure will be more fully understood from the following detailed description of the examples thereof, taken together with the drawings, in which:

FIG. 1 is a partly pictorial, partly block diagram view of a phacoemulsification system constructed and operative in accordance with some examples of the disclosure;

FIG. 2A is pictorial view of a tip of a phacoemulsification probe inside a patient's eye, in accordance with examples of the disclosure;

FIG. 2B is pictorial view of a tip of a phacoemulsification probe inside a patient's eye, in accordance with examples of the disclosure;

FIG. 2C is pictorial view of a tip of a phacoemulsification probe inside a patient's eye, in accordance with examples of the disclosure;

FIG. 2D is pictorial view of a tip of a phacoemulsification probe inside a patient's eye, in accordance with examples of the disclosure;

FIG. 3A is a flowchart of steps in a method for avoiding vacuum surge situations during an operation, in accordance with examples of the disclosure; and

FIG. 3B is a flowchart of steps in a method for avoiding vacuum surge situations during an operation, in accordance with examples of the disclosure.

### DETAILED DESCRIPTION OF EXAMPLES

In the following description, numerous specific details are set forth in order to provide a thorough understanding of the present disclosure. It will be apparent to one skilled in the art, however, that the present disclosure may be practiced without these specific details. In other instances, well-known circuits, control logic, and the details of computer program instructions for conventional algorithms and processes have not been shown in detail in order not to obscure the present disclosure unnecessarily.

Software programming code, which embodies aspects of the present disclosure, is typically maintained in permanent storage, such as a computer readable medium. In a client-server environment, such software programming code may be stored on a client or a server. The software programming code may be embodied on any of a variety of known media for use with a data processing system. This includes, but is not limited to, magnetic and optical storage devices such as disk drives, magnetic tape, compact discs (CD's), digital video discs (DVD's), and computer instruction signals embodied in a transmission medium with or without a carrier wave upon which the signals are modulated. For example, the transmission medium may include a communications network, such as the Internet. In addition, while the disclosure may be embodied in computer software, the functions necessary to implement the disclosure may alternatively be embodied in part or in whole using hardware components such as application-specific integrated circuits or other hardware, or some combination of hardware components and software.

In the description below, the term “about” as related to numerical values may include values that are in the range of +/−10% of the indicated value.

### Overview

A cataract is often handled by a phacoemulsification procedure, in which an ultrasonic handpiece is used to insert a needle into the patient's eye, wherein the tip of the needle can vibrate at ultrasonic frequency, to emulsify the cataract.

The vibrations cause particles of the lens to be released from the lens. The particles are typically aspirated via an aspiration line running through or coupled with the needle, together with fluids.

However, some particles may be too large, or reach the inlet of the aspiration line at an unsuitable angle, and may thus occlude the inlet for a while.

As a result, the vacuum within the aspiration line increases, and when the particle is finally aspirated or is otherwise released from the inlet, the high vacuum in the line leads to a vacuum surge, with potentially traumatic consequences to the eye. High vacuum may also develop in other situations, for example after the tip of the needle is inserted into the eye and once it fully penetrates through the lens.

To avoid trauma to the eye, an Anti-Vacuum Surge (AVS) system or chamber stabilization system (CSS) or valve (collectively, “AVS system”) is activated, in order to stop the vacuum surge as soon as the occlusion is detected or is released. For example, the AVS system may be activated when a particle occluding the aspiration line is identified, or when it is eventually aspirated into the aspiration line. In another example, the AVS system may be activated when a particle occluding the aspiration line is broken into smaller pieces by applying vibrations, thereby releasing the occlusion. In yet another example, when the needle penetrates the lens the vacuum may increase, therefore the AVS system may also be activated to avoid possible damage to the eye due to a vacuum surge. In yet another example, the AVS system may also be activated when the cornea starts to collapse due to the vacuum surge. A collapsing cornea may be identified using a classifier trained upon a labeled collection of collapsed and non-collapsed corneas. Additionally, or alternatively, a collapsing cornea may be identified by a significant change in the appearance of the cornea, for example a change in at least a predetermined part of pixels in two images of the cornea.

A significant challenge is to identify as early as possible an actual or potential vacuum surge, such that the AVS system can be timely activated before the vacuum surge damages the eye.

Since phacoemulsification may be performed under an electronic microscope, it may be beneficial to process images captured by the microscope, to continuously detect whether an aspiration-stopping, a vacuum surge criteria or a valve-activation criteria (collectively referred to as “aspiration-stopping criteria”) has been met, and to activate the AVS system.

Thus, in some examples, a particle may be occluding the inlet of the needle, for example is at the same location adjacent to the inlet of the needle for a predetermined period of time, as demonstrated by the particle being captured and identified at the same location on two or more consecutive images captured by the microscope, it may be determined that the particle is occluding the inlet of the needle. In such situation, the particle may be emulsified into smaller pieces by applying ultrasound waves, immediately followed by activating the AVS system, resulting in minimizing or eliminating vacuum surge which may damage the eye. In some examples, the AVS system may be activated without operating the ultrasound, such that the particle will move away from the inlet and may later reach the inlet at a different direction or angle that would allow it to be aspirated.

Thus, if it is detected by analyzing the images that the particle is occluding the inlet, once the particle is released, for example is emulsified, the AVS system may be activated to avoid a vacuum surge.

The occlusion of the tip by a particle may be identified by analyzing one or more images, and its release may be identified by analyzing further one or more images, in a number of scenarios. One such scenario may be recognized when the tip of the needle is hidden by a particle for a predetermined period of time, and is later exposed, which may occur since the particle has been emulsified. Another scenario may be when the tip of the needle, a particle, and a relative position therebetween are stable, for example for a predetermined period of time, followed by a change in either the particle (including disappearing, change of size, location, or shape) or the relative position between the particle and the tip.

In another example, high vacuum may develop when the needle penetrates the lens. The vacuum may thus increase, and may later be released at once, causing a vacuum surge which may be harmful to the eye. Thus, when it is identified by analyzing two or more images that the needle has penetrated the lens, aspiration may be stopped, and reactivated at a later time.

In yet another example, as the cornea may collapse under the vacuum surge, once a change in the appearance of the cornea is detected, the aspiration may be deactivated or stopped to avoid the vacuum surge.

In further examples, the vacuum may be sensed, for example by a sensor (e.g., pressure or flow), and aspiration may be deactivated or stopped subject to the combination of identifying from the captured images that one of the aspiration-stopping criteria as exemplified above is met, and the vacuum as sensed by the sensor is changing abruptly (e.g., drop in vacuum). Activating the AVS system only when the two conditions are met may reduce the number of false alarms, and may avoid ineffective or harmful aspiration deactivation or stopping.

It is appreciated that further criteria may be defined for identifying situations in which it is required to stop the aspiration in order to avoid the vacuum surge.

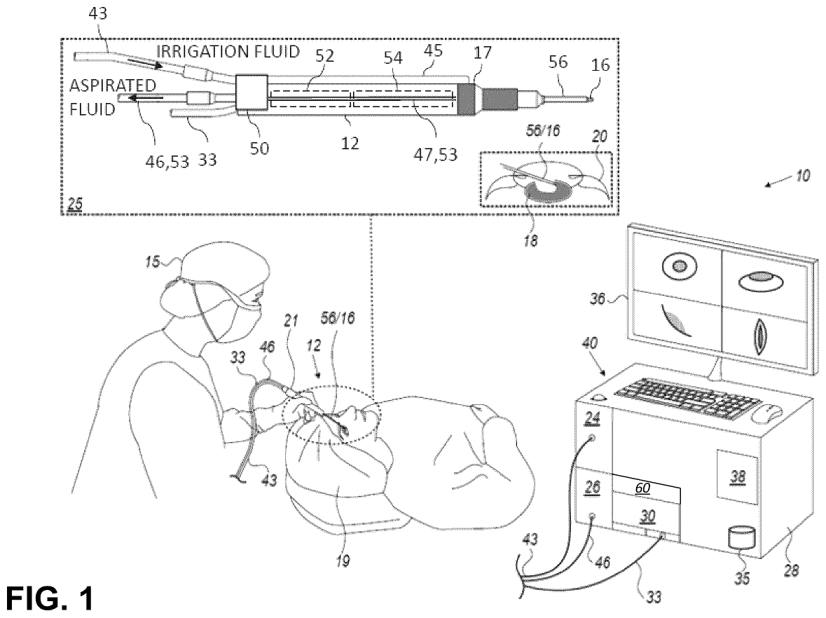

Reference is now made to FIG. 1 that is a partly pictorial, partly block diagram view of a phacoemulsification system 10 constructed and operative in accordance with some exemplary examples of the present disclosure.

Phacoemulsification system 10 comprises a phacoemulsification probe 12 (e.g., a handpiece). In some embodiments, phacoemulsification probe 12 may be replaced by any suitable medical tool. As seen in the pictorial view of system 10, and in inset 25, phacoemulsification probe 12 comprises a needle 16, a probe body 17, and a coaxial irrigation sleeve 56 that at least partially surrounds needle 16 and creates a fluid pathway between the external wall of the needle and the internal wall of the irrigation sleeve, where needle 16 is hollow to provide an aspiration channel. Moreover, irrigation sleeve 56 may have one or more side ports at, or near, the distal end to allow irrigation fluid to flow towards the distal end of the phacoemulsification probe 12 through the fluid pathway and out of the port(s). Needle 16 of the phacoemulsification probe 12 is configured for insertion into a lens capsule 18 of an eye 20 of a patient 19 by a physician 15, to remove a cataract. While needle 16 (and irrigation sleeve 56) are shown in inset 25 as a straight object, any suitable needle may be used with phacoemulsification probe 12, for example, a curved or bent tip needle commercially available from Johnson & Johnson Surgical Vision, Inc., Irvine, CA, USA. In the example of FIG. 1, during the phacoemulsification procedure, a pumping subsystem 24 comprised in a console 28 pumps irrigation fluid from an irrigation reservoir (not shown) to irrigation sleeve 56 to irrigate eye 20. The irrigation fluid is pumped via an irrigation tubing line 43 running from console 28 to an irrigation channel 45 of probe 12, the distal end of irrigation channel 45 including the fluid pathway in irrigation sleeve 56. In another example, pumping sub-system 24 may be coupled or replaced with a gravity fed irrigation source such as a balanced salt solution bottle/bag. Eye fluid and waste matter (e.g., emulsified parts of the cataract) are aspirated via an aspiration channel 47, which extends from the hollow of needle 16 through phacoemulsification probe 12, and then via an aspiration tubing line 46 to a collection receptacle (not shown) in console 28. The aspiration is affected by a pumping sub-system 26, also comprised in console 28. Aspiration tubing line 46 and aspiration channel 47 are collectively referred to as an aspiration line 53. Aspiration line 53 is therefore partially disposed in needle 16 and is coupled with pumping sub-system 26, which is configured to remove the fluid and waste matter from eye 20 via aspiration line 53.

System 10 may include an AVS system 50 (which in an example, may be removable), which may include one or more valves to regulate the flow of fluid in irrigation channel 45 and/or aspiration line 53 as well as sensors. Parts of irrigation channel 45 and aspiration line 53 are disposed in probe body 17 and parts are disposed in AVS 50. AVS system 50 may also be external to the handpiece but fluidly coupled with the aspiration line 53, irrigation channel 45, and irrigation line 43, e.g., proximal to the proximal end of handpiece 12. Phacoemulsification probe 12 may include other elements, such as an ultrasonic actuator 52, e.g., piezoelectric crystal, coupled with a horn 54 configured to support needle 16 and drive vibration of needle 16 to emulsify the lens of eye 20. Ultrasonic actuator 52 is configured to vibrate needle 16 in a resonant vibration mode. The vibration of needle 16 is used to break a cataract into small pieces during a phacoemulsification procedure.

Console 28 may comprise an ultrasonic (e.g., piezoelectric) drive module 30, coupled with ultrasonic actuator 52, using electrical wiring running in a cable 33. Drive module 30 is controlled by a controller 38 and conveys processor-controlled driving signals via cable 33 to, for example, maintain needle 16 at maximal vibration amplitude. Drive module 30 may be realized in hardware or software, for example, in a proportional-integral-derivative (PID) control architecture. Controller 38 may also be configured to receive signals from sensors in phacoemulsification probe 12 and control one or more valves to regulate the flow of fluid in irrigation channel 45 and/or aspiration line 53. In some examples, at least some of the functionality of controller 38 may be implemented using a controller disposed in phacoemulsification probe 12 (e.g., in cartridge 50).

Controller 38 may receive user-based commands via a user interface 40, which may include setting a vibration mode and/or frequency of ultrasonic actuator 52, and setting or adjusting an irrigation and/or aspiration rate of pumping sub-systems 24/26. In some examples, user interface 40 and a display 36 may be combined as a single touch screen displaying a graphical user interface. In some examples, physician 15 may use a foot pedal (not shown) as a means of control. Additionally, or alternatively, controller 38 may receive the user-based commands from controls located in a handle 21 of probe 12.

Some or all of the functions of controller 38 may be combined in a single physical component or, alternatively, implemented using multiple physical components. These physical components may comprise hard-wired or programmable devices, or a combination of the two. In some examples, at least some of the functions of controller 38 may be carried out by suitable software stored in a memory device 35. This software may be downloaded to a device in electronic form, over a network, for example. Alternatively, or additionally, the software may be stored in tangible, non-transitory computer-readable storage media, such as optical, magnetic, or electronic memory.

Console 28 may comprise one or more processors 60, configured to perform analyses and computations, including but not limited to image analysis of images captured by a stereomicroscope. Thus processor(s) 60 may execute analyses including image analyses to identify needle 16 and/or a tip thereof within eye 20, to identify particles, to determine whether particles seen in two images are the same particle, to combine image analyses with aspiration values (e.g., pressure or vacuum) measured within an aspiration line, or the like. However, in further examples, some or all of the computations and in particular the image analysis may be performed by another computing platform being in communication with processor(s) 60.

The system shown in FIG. 1 may include further elements which are omitted for clarity of presentation. For example, physician 15 typically performs the procedure using a stereomicroscope. Physician 15 may use other surgical tools in addition to probe 12, which are also not shown, in order to maintain clarity and simplicity of presentation.

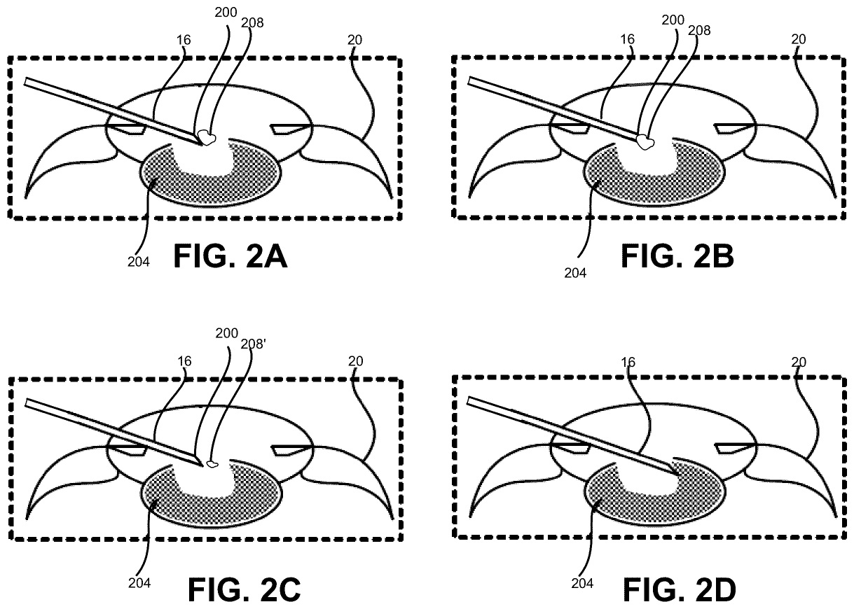

Reference is now made to FIGS. 2A-2D, showing some examples of scenarios in which an AVS system may be deactivated or stopped. FIGS. 2A and 2B show schematic illustrations of needle 16 inside eye 20 of patient 19. As shown in FIGS. 2A and 2B, the light sensor may capture sequences of images depicting tip 200 and particle 208 released from lens 204 during the operation. Using image analysis techniques, the shape of particle 208 and the position of tip 200 relative to particle 208 may be determined and monitored.

Thus, FIG. 2A shows particle 208 being adjacent to tip 300 and occluding tip 200, but without hiding tip 200. In FIG. 2B, however, particle 208 is attached to tip 200 and hides tip 200.

The situations in each of FIGS. 2A and 2B may be captured in one or more consecutive frames. In some examples, if the relative position between tip 200 (if visible) and particle 208 is the same in two or more consecutive frames, it may be assumed that particle 208 is occluding the inlet of tip 200, and the vacuum within the aspiration line may be increasing.

When an occlusion situation occurs, a user, such as a physician may recognize the situation when examining the lens and tip 200 through the microscope. The user may then operate the ultrasound transducer to emulsify particle 208 and break it into smaller parts that may be aspirated.

FIG. 2C shows particle 208′ which may be a sub-part of particle 208 and may be aspirated into tip 200. However, in some examples, no particle such as 208 or 208′ is visible in the image, as particle 208 or its subparts were aspired or drifted away.

Thus, one scenario as shown in FIGS. 2A and 2C is that the particle first occluded the inlet and was then released causing a change in the relative position between the particle and the tip, and another scenario as shown in FIGS. 2B and 2C is that tip 200 was hidden by the particle and then exposed.

In the two scenarios, the situation may have a static phase in which the tip of the needle is occluded for a predetermined period of time, such that, for example, one, two or more frames such as FIG. 2A or FIG. 2B may be captured, followed by a frame such as FIG. 2C in which the particle is released (or has disappeared). Thus, it is required as soon as the situation is identified to activate the AVS system, for example by closing the valve, and stop the aspiration, to avoid vacuum surge and damage to the eye once the particle is no longer occluding the inlet.

FIG. 2D demonstrates another situation, in which needle 16 penetrates lens 204, which may create a vacuum surge, that when released may traumatize the eye. Thus, when it is realized by processing images captured by the stereomicroscope that needle 16 has just penetrated lens 204, the aspiration may be deactivated or stopped, thus avoiding a vacuum surge. The situation may be identified, for example, by recognizing the tip of needle 16 at a distance from lens 204 in one frame, and within lens 204 in a following frame.

A further situation may relate to identifying that the shape of the cornea if the eye is distorting since the cornea is starting to collapse due to excessive aspiration, at which point it is required to stop the aspiration.

It is appreciated that the situations detailed above, and possibly additional situations, may be identified as meeting an aspiration-stopping criteria, optionally in combination with identifying a vacuum surge, for example by an aspiration sensor within aspiration line 53.

In some examples, the frames may be captured by a light sensor which can be positioned in proximity to eye 20 as part of the stereomicroscope. The images may be captured under illumination from a light source which may be located at the distal end of needle 16 or located on a second instrument placed inside the eye. The frames may be captured at a high frame rate, such as about 60-200, for example 120 frames per second, thereby fast processing thereof can provide valuable information.

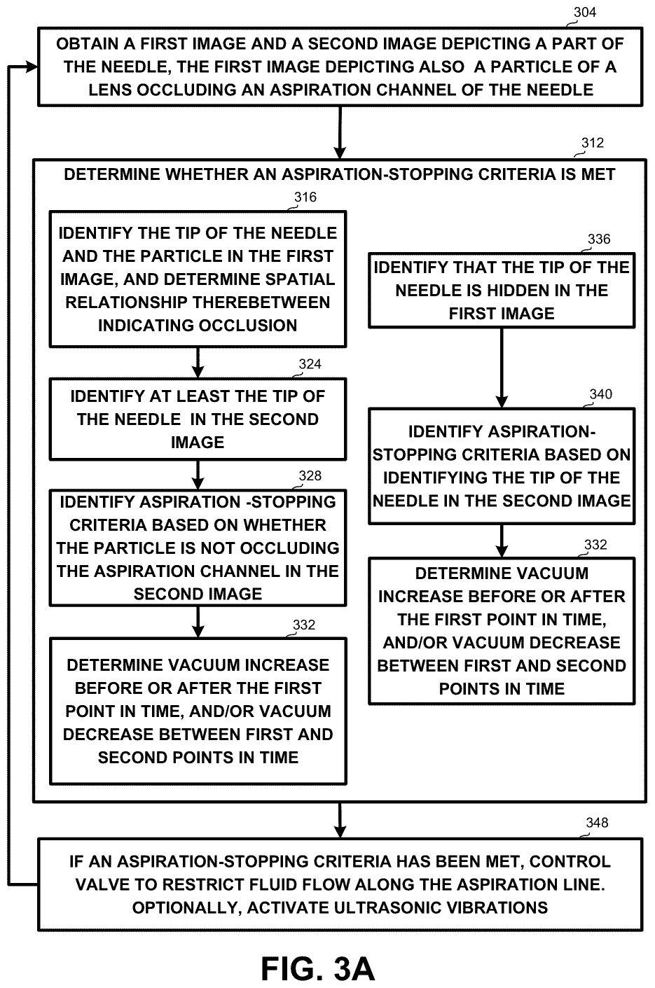

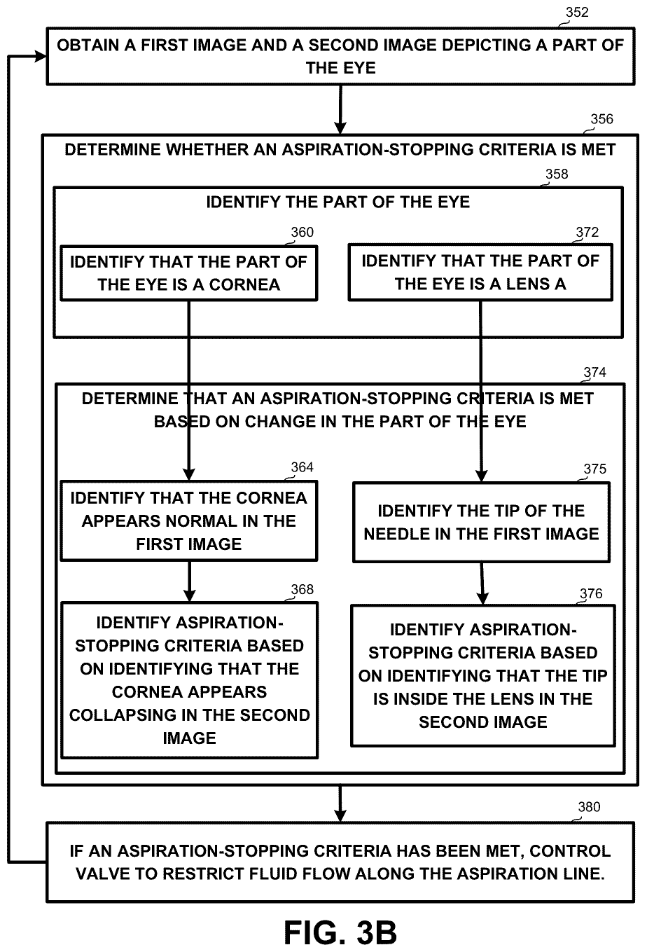

Referring now to FIGS. 3A and 3B, showing flowcharts of steps in methods for avoiding or mitigating vacuum surge situations during an operation, in accordance with some examples of the disclosure. The flowcharts describe that a sequence of images is captured, each depicting one or more of the tip or another part of the needle, a particle, the lens, the cornea, or other parts of the eye. The images are compared to each other or to predetermined structures, to identify based on spatial relationship between the captured elements, or on the structure of the eye organs, a situation of a possible vacuum surge. Once such situation is identified, the AVS system may be activated to avoid or mitigate the vacuum surge.

Referring now to FIG. 3A, showing a flowchart of steps in a first exemplary method for avoiding or mitigating vacuum surge situations during an operation, in accordance with some examples of the disclosure.

At step 304, a first image and a second image (also referred to as frames) may be obtained, which depict at least part of a needle of the probe, the part of the needle being within the eye. In some examples, the first image may also depict a particle of the lens of the eye wherein the particle may have been released from the lens. The first image may also depict that the particle is fully or partially occluding the aspiration line of the needle.

The images may be obtained by a stereomicroscope used by a user such as a physician performing the operation, for example a phacoemulsification cataract surgery. The first image may be captured at a first point in time, the second image may be captured at a second point in time later than the first point in time. It is appreciated that some of the steps below may be performed prior to the capturing of the second image, i.e., processing may start on the first image before the second image is captured. In some embodiments, the first image may be a part of a first set comprising one or more images.

At step 312, it may be determined based upon the first and second images whether a known aspiration-stopping criterion is met. It may be determined, for example, whether the part of the needle, the particle, and the relative position therebetween indicate that the particle is occluding the needle and is then released.

In a first example, an aspiration-stopping criteria of a particle occluding the inlet may be identified.

At step 316, the particle and the part of the needle may be identified within the first image. The part of the needle may be a tip of the needle which may or may not be marked or colored in a distinguishable manner, or a more distal part of the needle, for example if the tip is hidden by the particle. The part may be identified using an artificial intelligence (AI) engine, such as a classifier trained upon a plurality of labeled images to identify the part. Additionally, or alternatively, the part may be identified using any other technique such as pattern matching. The particle may also be identified using an AI engine trained to identify particles that are typically released during phacoemulsification operations. As above, any other image analysis technique which may differentiate a particle from its background may be used.

Once the particle and the tip are identified, the spatial relationship therebetween may be determined to indicate that the particle is fully or partially occluding the aspiration line of the needle in the first image, wherein an occlusion may be determined based upon the particle being adjacent to the inlet of the aspiration line.

If the first image is comprised in a first set of images, then an occlusion may also be determined by the tip of the needle, the particle, and the spatial relationship being the same in at least two consecutive images of the first set of images.

It is appreciated that if the first image is included in a first set of images, then step 316 may include determining that the particle in at least two images of the first set of images is the same particle, for example based on its shape. At step 324, the tip of the needle may be identified in the second image, similarly, to step 316 above.

At step 328, it may be identified whether an aspiration-stopping criteria is met due to the particle not occluding the aspiration line in the second image, based upon the spatial relationship between the tip and the particle differing from the spatial relationship in the first image. The difference may be due to the particle being absent from the second image, if another particle (or multiple particles) which may be a sub-part of the particle of the first image is present at the vicinity of the tip, or the particle has moved relative to the tip of the needle. The change in the spatial relationship may be due to the application of ultrasonic vibrations applied to the particle and emulsifying it.

The situation identified at step 328 may thus be an aspiration-stopping criteria, where after the particle has been removed from the inlet of the needle and is thus not occluding the aspiration line of the needle anymore, a vacuum surge needs to be avoided.

In some examples, at optional step 332 it may be determined that the vacuum within the aspiration line has increased in a time period immediately preceding or following the first point in time, and/or decreased between the first point in time and the second point in time.

In some examples, small deviations in the IOP from the target IOP may have no effect on the well-being of the eye. Therefore, such small variations from the target IOP may be eliminated from the computation. Thus, the increase and decrease may be considered provided the measured values exceed predetermined thresholds. Minor deviations of the measured values from the target IOP are not interpreted as increasing or decreasing values. The vacuum changes may be determined upon values received from a vacuum sensor located for example within aspiration line 53. The combination of determining relative position between the inlet of the needle and the particle, and the vacuum changes may reinforce the determination that an aspiration-stopping criteria has been met.

It is appreciated that steps 304, 312 (whether through steps 316, 324, 328 and optionally 332, or through steps 336, 340 and optionally 332 detailed below) and 348 detailed below may be repeated throughout the operation, to keep identifying these scenarios or others. Moreover, the second image of one iteration may be used as the first image for the next iteration.

It is appreciated that the steps above may be performed in a different order or at the same time. For example, step 332 may be performed before or at the same time as step 328 or before or at the same time as step 324.

For identifying another aspiration-stopping criteria, at step 336 it may be identified that the tip of the needle is hidden in the first image. If the first image is comprised in a first set of images, it may also be determined that the tip is hidden in the same manner in at least two consecutive images of the first set of images, for example the same part of the needle is hidden, by the same particle.

At step 340, the tip of the needle may be identified in the second image, meaning that the tip has been exposed between the first point in time and the second point in time, for example by ultrasonic vibrations applied to the particle and emulsifying it.

The situation identified at step 340 may thus be an aspiration-stopping criteria, where after the particle has been removed from the inlet of the needle, a vacuum surge needs to be avoided.

In some examples, at optional step 332 the vacuum changes as detailed above may be used in combination with the image processing to determine the occurrence of a vacuum-surge criteria.

If one of the aspiration-stopping criteria as identified using image analysis and optionally vacuum changes has been met, at step 348 an aspiration-stopping action may be taken for deactivation or stopping the aspiration, such as activating the AVS system to restrict fluid flow along the aspiration line (e.g., closing the aspiration line via a valve), thereby stopping the vacuum surge.

It is appreciated that the timing of taking the aspiration-stopping action 348 may be learned upon a plurality of cases, including frame sequences taken during previous operations, showing the particles and the needle, the time at which the AVS system was activated, and/or labels indicating whether the eye was damaged or not as a result of the aspiration. Thus, it would be beneficial to identify that the tip is occluded as soon as possible to reduce the vacuum surge development, and to activate the AVS system as early as possible after the ultrasound transducer has been activated.

Referring now to FIG. 3B, showing a flowchart of steps in a method for avoiding further vacuum surge situations during an operation, in accordance with some examples of the disclosure.

At step 352, a first image of at least a part of the eye may be captured at a first point in time, and a second image of the part of the eye may be captured at a second point in time later than the first point in time. Capturing may be performed as detailed above in association with step 304 of FIG. 3A.

At step 356, it may be determined based upon at least the part of the eye as depicted in the first image and second image, whether an aspiration-stopping criterion is met, and in particular a change in a part of the eye between the first image and the second image.

Step 356 may comprise step 358 for determining the part of the eye, and step 374 for determining that an aspiration-stopping criteria is met based upon a change in the part of the eve between the first and second images.

In a first example, an aspiration-stopping criteria of a collapsing cornea may be identified.

At step 360, the part of the eye may be located in the first image and in the second image, and identified as the cornea.

At step 364, it may be determined that the cornea appears normal in the first image. The determination may be performed by an engine such as a classifier trained upon a plurality of labeled images of normal and abnormal corneas. For example, a part of the abnormal images may be labeled as “cornea collapsing due to vacuum surge”.

At step 368, it may be identified that an aspiration-stopping criteria is met based upon the cornea appearing abnormal in the second image, for example it appears as if it is collapsing due to a developing vacuum surge.

The situation identified at step 368 may thus be an aspiration-stopping criteria, where upon identifying that the cornea is starting to collapse, the vacuum surge needs to be avoided.

For identifying another aspiration-stopping criteria, at step 372 it may be identified that in the first image the part of the eye is a lens.

At step 375, the tip of the needle may be identified in the first image, thus, the tip of the needle is not inside the lens.

At step 376, it may be identified that an aspiration-stopping criteria is met based upon the tip of the needle has penetrated the lens, for example the tip of the needle is not detected in the second image, while another part of the needle is still in the vicinity of the lens.

If one of the aspiration-stopping criteria as identified using image analysis has been met at step 368 or step 376, then at step 380, an aspiration-stopping action may be taken for deactivation or stopping the aspiration, such as activating the AVS system to restrict fluid flow along the aspiration line, thereby stopping the vacuum surge.

It is appreciated that the timing for taking the aspiration-stopping action 380 may be learned upon a plurality of cases, including frame sequences taken during previous operations, showing the part of the eye, the time at which the AVS system was activated, and labels indicating whether the eye was damaged or not as a result of the aspiration. Thus, it would be beneficial to identify that the tip is inserted into the lens or that the cornea is collapsing, and to activate the AVS system as early as possible to reduce the vacuum surge development. Such information may also be deduced from the frame sequences used for training the engine.

It is appreciated that steps 352, 356 (whether through steps 360, 364 and 368, or through steps 372 and 376) and 380 may be repeated throughout the operation, to keep identifying these scenarios or others. Moreover, the second image of one iteration may be used as the first image for the next iteration.

In some examples, the determination of which criteria of those detailed in FIG. 3A and FIG. 3B and/or additional ones, is to be searched for may depend on the stage within the operation, which may be tracked, indicated by a user, or the like. In other examples, a plurality of criteria, for example the occurrence of one or more of the criteria mentioned above or additional ones may be checked in parallel.

It is appreciated that the steps and modules disclosed above are in addition to the software, hardware, firmware, or other modules required for operating the probe, displaying the phacoemulsification process, performing other calculations required for example for operating irrigation pump 24 or aspiration pump 26, or the like.

The methods of FIGS. 3A and 3B may be coded as a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembly instructions, instruction-set-architecture instructions, machine instructions, machine dependent instructions, microcode, firmware instructions, state-setting data, or either source code or object code written in any combination of one or more programming languages, programming languages such as Java, C, C++, Python, or others. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some examples, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to examples of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### EXAMPLES

### Example 1

A phacoemulsification system, comprising: a phacoemulsification probe (12) having a needle (16) at its distal end, the needle (16) configured to be inserted into an eye (20) of a patient (19), the phacoemulsification probe (12) comprising an ultrasonic transducer; an aspiration line (53), wherein the aspiration line (53) is fluidly coupled with the needle (16); an Anti-Vacuum Surge (AVS) (50) system configured to control fluid flow in the aspiration line (53); and a processor (60), configured to: obtain (304) a first set of images, the first set of images comprising at least a first image captured at a first point in time, the first image depicting at least a part of the needle (16) and a particle of a lens of the eye occluding an aspiration line of the needle (16); obtain (304) a second image depicting at least the part of the needle (16), the second image captured at a second point in time later than the first point in time; determine (312) based on the first image and the second image whether an aspiration-stopping criteria is met; and subject to the aspiration-stopping criteria being met, control (348) the AVS system to restrict fluid flow along the aspiration line.

### Example 2

The phacoemulsification system according to example 1, wherein the aspiration-stopping criteria is at least one of: an occlusion of the aspiration line (53) by a particle has been released; the needle (16) penetrating the lens (204); and a cornea of the eye (20) collapsing.

### Example 3

The phacoemulsification system according to any of examples 1-2, further comprising a sensor configured to provide a signal indicative of vacuum in the aspiration line (53), and wherein the determining (312) whether the aspiration-stopping criteria is met further comprises detecting (332) an increase in the signal preceding or following the first point in time, or a decrease in the signal between the first point in time and the second point in time.

### Example 4

The phacoemulsification system according to any of examples 1-3, wherein determining (312) whether the aspiration-stopping criteria is met comprises: identifying (316) the part of the needle and the particle in the first image, wherein the part of the needle is a tip of the needle; determining (316) a spatial relationship between the tip of the needle and the particle in the first image; identifying (324) at least the tip of the needle in the second image; and determining (328) that the aspiration-stopping criteria is met upon identifying a second particle different from the particle, or a different spatial relationship between the tip of the needle and the particle, between the first image and the second image, indicating that the particle is not occluding the aspiration line in the second image.

### Example 5

The phacoemulsification system according to any of examples 1-4, wherein determining (312) that the aspiration-stopping criteria is met further comprises: identifying (336) that the part of the needle (16) being a tip of the needle (16) in hidden by the particle in the first image; and based on identifying that the tip of the needle (16) is not hidden by the particle in the second image, determining (340) that the aspiration-stopping criteria is met.

### Example 6

The phacoemulsification system according to any of examples 1-5, wherein the processor (60) is configured to perform repeatedly said obtaining (304) the first image and the second image, said determining (312) and said controlling (348).

### Example 7

The phacoemulsification system according to examples 1-6, wherein determining (312) that the aspiration-stopping criteria is met further comprises determining that a spatial relationship between the part of the needle (16) and the particle is identical in at least two images from the first set of images.

### Example 8

The phacoemulsification system according to examples 1-7, wherein the aspiration line (53) is at least partially disposed in the needle (16).

### Example 9

A method for applying phacoemulsification to an eye, comprising: providing an ophthalmic surgical system comprising a phacoemulsification probe (12) having a needle (16) at its distal end, the needle (16) configured to be inserted into an eye (20) of a patient (19), the phacoemulsification probe (12) comprising an ultrasonic transducer, an aspiration line (53), wherein the aspiration line (53) is fluidly coupled with the needle (16), an Anti-Vacuum Surge (AVS) (50) system configured to control fluid flow in the aspiration line (53); and a processor (60); obtaining (304) a first set of images, the first set of images comprising at least a first image captured at a first point in time, the first image depicting at least a part of the needle and a particle of a lens (204) of the eye (20) occluding an aspiration line (53) of the needle; obtaining (304) a second image depicting at least the part of the needle (16), the second image captured at a second point in time later than the first point in time; determining (312) based on the first image and the second image whether an aspiration-stopping criteria is met; and subject to the aspiration-stopping criteria being met, control (348) the AVS system (50) to restrict fluid flow along the aspiration line (53).

### Example 10

The method according to example 9, wherein the aspiration-stopping criteria is at least one of: an occlusion of the aspiration line (53) by a particle has been released; the needle (16) penetrating the lens (204); and a cornea of the eye (20) collapsing.

### Example 11

The method according to any of examples 9-10, wherein determining (312) whether the aspiration-stopping criteria is met further comprises detecting (332) an increase in a signal indicative of vacuum in the aspiration line preceding or following the first point in time, or a decrease in the signal between the first point in time and the second point in time, wherein the signal is provided by a sensor.

### Example 12

The method according to any of examples 9-11, wherein determining (312) whether the aspiration-stopping criteria is met comprises: identifying (316) the part of the needle and the particle in the first image, wherein the part of the needle is a tip of the needle; determining (316) a spatial relationship between the tip of the needle and the particle in the first image; identifying (324) at least the tip of the needle in the second image; and determining (328) that the aspiration-stopping criteria is met upon identifying a second particle different from the particle, or a different spatial relationship between the tip of the needle and the particle, between the first image and the second image, indicating that the particle is not occluding the aspiration line (53) in the second image.

### Example 13

The method according to any of examples 9-12, wherein determining (312) that the aspiration-stopping criteria is met further comprises: identifying (336) that the part of the needle being a tip of the needle (16) in hidden by the particle in the first image; and based on identifying (340) that the tip of the needle (16) is not hidden by the particle in the second image, determining that the aspiration-stopping criteria is met.

### Example 14

The method according to any of examples 9-13, wherein said obtaining (304) the first image and the second image, said determining (312) and said controlling (348) are performed repeatedly.

### Example 15

The method according to any of examples 9-14, wherein determining3 (312) that the aspiration-stopping criteria is met further comprises determining that a spatial relationship between the part of the needle and the particle is identical in at least two images from the first set of images.

### Example 16

The method according to any of examples 9-15, wherein the aspiration line (53) is at least partially disposed in the needle (16).

### Example 17

A phacoemulsification system, comprising: a phacoemulsification probe (12) having a needle (16) at its distal end, the needle configured to be inserted into an eye (20) of a patient (19), the phacoemulsification probe (12) comprising an ultrasonic transducer; an aspiration line (53), wherein the aspiration line (53) is fluidly coupled with the needle; an Anti-Vacuum Surge (AVS) (50) system configured to control fluid flow in the aspiration line; and a processor (60), configured to: obtain (352) a first image captured at a first point in time and a second image captured at a second point in time later than the first point in time; identify (358) a part of the eye in the first image and the second image; determine (374) based upon at least the part of the eye appearing different between the first image and the second image, that an aspiration-stopping criteria is met; and subject to identifying that the aspiration-stopping criteria is met, control (380) the AVS system (50) to restrict fluid flow along the aspiration line.

### Example 18

The phacoemulsification system according to example 17, wherein determining (356) that the aspiration-stopping criteria is met comprises: identifying that the part of the eye (20) is a cornea (360); identifying (364) that the cornea appears normal in the first image; and identifying (368) that the cornea appears collapsing in the second image.

### Example 19

The phacoemulsification system according to any of examples 17-18, wherein determining that the aspiration-stopping criteria is met comprises: identifying that the part of the eye (20) is a lens (204); identifying that a tip of a needle (16) is not inserted into the lens (204) in the first image (375); and identifying (376) that a tip of a needle (16) is inserted into the lens (204) in the second image.

### Example 20

The phacoemulsification system according to any of examples 17-19, wherein determining that the aspiration-stopping criteria is met further comprises detecting an increase in a signal indicative of vacuum in the aspiration line preceding or following the first point in time, or a decrease in the signal between the first point in time and the second point in time, wherein the signal is provided by a sensor.

### Example 21

A method for applying phacoemulsification to an eye, comprising: providing an ophthalmic surgical system comprising a phacoemulsification probe (12) having a needle (16) at its distal end, the needle (16) configured to be inserted into an eye (20) of a patient (19), the phacoemulsification probe (12) comprising an ultrasonic transducer, an aspiration line (53), wherein the aspiration line (53) is fluidly coupled with the needle, an Anti-Vacuum Surge (AVS) system configured to control fluid flow in the aspiration line, and a processor (60); obtaining (352) a first image captured at a first point in time and a second image captured at a second point in time later than the first point in time; identifying (358) a part of the eye in the first image and the second image; determine (374) based upon at least the part of the eye appearing different between the first image and the second image, that an aspiration-stopping criteria is met; and subject to identifying that the aspiration-stopping criteria is met, controlling (380) the AVS system (50) to restrict fluid flow along the aspiration line.

### Example 22

A computer program product comprising a non-transitory computer readable medium retaining program instructions, which instructions when read by a processor (60), cause the processor (60) to perform: obtaining (304) a first set of images, the first set of images comprising at least a first image captured at a first point in time, the first image depicting at least a part of a needle (16) comprised at a distal end of a phacoemulsification probe (12) of an phacoemulsification system, and a particle (208) of a lens (204) of the eye (20) of a patient (19) operated upon with the phacoemulsification system, the particle (208) occluding an aspiration line (53) of the needle (16); obtaining a second image depicting at least the part of the needle (16), the second image captured at a second point in time later than the first point in time; determining (312) based on the first image and the second image whether an aspiration-stopping criteria is met; and subject to the aspiration-stopping criteria being met, controlling (348) an Anti-Vacuum Surge (AVS) comprised in the phacoemulsification system to restrict fluid flow along the aspiration line (53).

### Example 23

A computer program product comprising a non-transitory computer readable medium retaining program instructions, which instructions when read by a processor (60), cause the processor (60) to perform: obtaining (352) a first image captured at a first point in time and a second image captured at a second point in time later than the first point in time, the first image and the second image captured during a phacoemulsification operation performed using a phacoemulsification system comprising: a phacoemulsification probe (12) having a needle (16) at its distal end, the needle (16) configured to be inserted into an eye (20) of a patient (19); and an Anti-Vacuum Surge (AVS) system (50); identifying (358) a part of the eye in the first image and the second image; determining (374) based upon at least the part of the eye appearing different between the first image and the second image, that an aspiration-stopping criteria is met; and subject to identifying that the aspiration-stopping criteria is met, controlling (380) the AVS system to restrict fluid flow along the aspiration line.

Although the examples described herein mainly address cardiac diagnostic applications, the methods and systems described herein can also be used in other medical applications.

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art.

1. A phacoemulsification system, comprising:
a phacoemulsification probe having a needle at its distal end, the needle configured to be inserted into an eye of a patient, the phacoemulsification probe comprising an ultrasonic transducer;
an aspiration line, wherein the aspiration line is fluidly coupled with the needle;
an Anti-Vacuum Surge (AVS) system configured to control fluid flow in the aspiration line; and
a processor, configured to:
obtain a first set of images, the first set of images comprising at least a first image captured at a first point in time, the first image depicting at least a part of the needle and a particle of a lens of the eye occluding an aspiration line of the needle;
obtain a second image depicting at least the part of the needle, the second image captured at a second point in time later than the first point in time;
determine based on the first image and the second image whether an aspiration-stopping criteria is met; and
subject to the aspiration-stopping criteria being met, control the AVS system to restrict fluid flow along the aspiration line.

2. The phacoemulsification system of claim 1, wherein the aspiration-stopping criteria is at least one of: an occlusion of the aspiration line by a particle has been released; the needle penetrating the lens; and a cornea of the eye collapsing.

3. The phacoemulsification system of claim 1, further comprising a sensor configured to provide a signal indicative of vacuum in the aspiration line, and wherein the determining whether the aspiration-stopping criteria is met further comprises detecting an increase in the signal preceding or following the first point in time, or a decrease in the signal between the first point in time and the second point in time.

4. The phacoemulsification system of claim 1, wherein determining whether the aspiration-stopping criteria is met comprises:
identifying the part of the needle and the particle in the first image, wherein the part of the needle is a tip of the needle;
determining a spatial relationship between the tip of the needle and the particle in the first image;
identifying at least the tip of the needle in the second image; and
determining that the aspiration-stopping criteria is met upon identifying a second particle different from the particle, or a different spatial relationship between the tip of the needle and the particle, between the first image and the second image, indicating that the particle is not occluding the aspiration line in the second image.

5. The phacoemulsification system of claim 1, wherein determining that the aspiration-stopping criteria is met further comprises:
identifying that the part of the needle being a tip of the needle in hidden by the particle in the first image; and
based on identifying that the tip of the needle is not hidden by the particle in the second image, determining that the aspiration-stopping criteria is met.

6. The phacoemulsification system of claim 1, wherein the processor is configured to repeatedly said obtaining the first image and the second image, said determining and said controlling.

7. The phacoemulsification system of claim 1, wherein determining that the aspiration-stopping criteria is met further comprises determining that a spatial relationship between the part of the needle and the particle is identical in at least two images from the first set of images.

8. The phacoemulsification system of claim 1, wherein the aspiration line is at least partially disposed in the needle.

9. A method for applying phacoemulsification to an eye, comprising:
providing an ophthalmic surgical system comprising a phacoemulsification probe having a needle at its distal end, the needle configured to be inserted into an eye of a patient, the phacoemulsification probe comprising an ultrasonic transducer, an aspiration line, wherein the aspiration line is fluidly coupled with the needle, an Anti-Vacuum Surge (AVS) system configured to control fluid flow in the aspiration line, and a processor;
obtaining a first set of images, the first set of images comprising at least a first image captured at a first point in time, the first image depicting at least a part of the needle and a particle of a lens of the eye occluding an aspiration line of the needle;
obtaining a second image depicting at least the part of the needle, the second image captured at a second point in time later than the first point in time;
determining based on the first image and the second image whether an aspiration-stopping criteria is met; and
subject to the aspiration-stopping criteria being met, controlling the AVS system to restrict fluid flow along the aspiration line.

10. The method of claim 9, wherein the aspiration-stopping criteria is at least one of: an occlusion of the aspiration line by a particle has been released; the needle penetrating the lens; and a cornea of the eye collapsing.

11. The method of claim 9, wherein determining whether the aspiration-stopping criteria is met further comprises detecting an increase in a signal indicative of vacuum in the aspiration line preceding or following the first point in time, or a decrease in the signal between the first point in time and the second point in time, wherein the signal is provided by a sensor.

12. The method of claim 9, wherein determining whether the aspiration-stopping criteria is met comprises:
identifying the part of the needle and the particle in the first image, wherein the part of the needle is a tip of the needle;
determining a spatial relationship between the tip of the needle and the particle in the first image;
identifying at least the tip of the needle in the second image; and
determining that the aspiration-stopping criteria is met upon identifying a second particle different from the particle, or a different spatial relationship between the tip of the needle and the particle, between the first image and the second image, indicating that the particle is not occluding the aspiration line in the second image.

13. The method of claim 9, wherein determining that the aspiration-stopping criteria is met further comprises:
identifying that the part of the needle being a tip of the needle in hidden by the particle in the first image; and
based on identifying that the tip of the needle is not hidden by the particle in the second image, determining that the aspiration-stopping criteria is met.

14. The method of claim 9, wherein said obtaining the first image and the second image, said determining and said controlling are performed repeatedly.

15. The method of claim 9, wherein determining that the aspiration-stopping criteria is met further comprises determining that a spatial relationship between the part of the needle and the particle is identical in at least two images from the first set of images.

16. The method of claim 9, wherein the aspiration line is at least partially disposed in the needle.

17. A phacoemulsification system, comprising:
a phacoemulsification probe having a needle at its distal end, the needle configured to be inserted into an eye of a patient, the phacoemulsification probe comprising an ultrasonic transducer;
an aspiration line, wherein the aspiration line is fluidly coupled with the needle;
an Anti-Vacuum Surge (AVS) system configured to control fluid flow in the aspiration line; and
a processor, configured to:
obtain a first image captured at a first point in time and a second image captured at a second point in time later than the first point in time;
identify a part of the eye in the first image and the second image;
determine based upon at least the part of the eye appearing different between the first image and the second image, that an aspiration-stopping criteria is met; and
subject to identifying that the aspiration-stopping criteria is met, control the AVS system to restrict fluid flow along the aspiration line.

18. The phacoemulsification system of claim 17, wherein determining that the aspiration-stopping criteria is met comprises:
identifying that the part of the eye is a cornea;
identifying that the cornea appears normal in the first image; and
identifying that the cornea appears collapsing in the second image.

19. The phacoemulsification system of claim 17, wherein determining that the aspiration-stopping criteria is met comprises:
identifying that the part of the eye is a lens;
identifying that a tip of a needle is not inserted into the lens in the first image; and
identifying that a tip of a needle is inserted into the lens in the second image.

20. The phacoemulsification system of claim 17, wherein determining that the aspiration-stopping criteria is met further comprises detecting an increase in a signal indicative of vacuum in the aspiration line preceding or following the first point in time, or a decrease in the signal between the first point in time and the second point in time, wherein the signal is provided by a sensor.
